(19) United States
(12) Patent Application Publication
(10) Pub. No.: US 2012/0220655 A1
(43) Pub. Date: Aug. 30, 2012

(54) CRYSTALLINE FORMS OF FESOTERODINE FUMARATE AND FESOTERODINE BASE

(75) Inventor: Peter Kerekes, Szerencs (HU)

(73) Assignee: TEVA GYOGYSZERGYAR ZARTKORUEN MUKODO RESZVENYTARSASAG, Debrecen (HU)

(21) Appl. No.: 13/394,136

(22) PCT Filed: Sep. 3, 2010
(86) PCT No.: PCT/US2010/047820

§ 371 Date: May 7, 2012

Related U.S. Application Data
(60) Provisional application No. 61/239,491, filed on Sep. 3, 2009.
(60) Provisional application No. 61/240,271, filed on Sep. 7, 2009.
(60) Provisional application No. 61/258,321, filed on Nov. 5, 2009.
(60) Provisional application No. 61/286,829, filed on Dec. 16, 2009.
(60) Provisional application No. 61/333,071, filed on May 10, 2010.

(51) Int. Cl. A61K 31/222; A61P 13/10; C07C 219/30

(52) U.S. Cl. 514/546; 560/129

(57) ABSTRACT

Polymorphically pure crystalline forms of fesoterodine fumarate and fesoterodine base are described and characterized.

### CROSS-REFERENCE TO RELATED APPLICATIONS

This application claims the benefit of U.S. Provisional Patent Application Ser. Nos. 61/239,491, filed Sep. 3, 2009, 61/240,271, filed Sep. 7, 2009, 61/258,321, filed Nov. 5, 2009, 61/286,829, filed Dec. 16, 2009, and 61/333,071, filed May 10, 2010, which are incorporated herein by reference.

### FIELD OF THE INVENTION

The present invention relates to crystalline forms of fesoterodine fumarate and fesoterodine base.

### BACKGROUND OF THE INVENTION

Fesoterodine fumarate is a selective M3 antimuscarinic agent indicated for the treatment of overactive bladder syndrome with symptoms of urgency, urinary incontinence and increased urinary frequency.

The chemical name of Fesoterodine fumarate is isobutyric acid 2-(3-(diiso-propylamino)-1-(R)-phenylpropyl)-4-(hydroxymethyl) phenyl ester hydrogenfumarate, having the following structure:

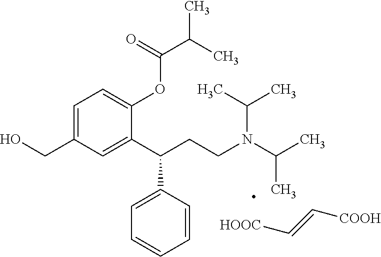

Fesoterodine fumarate is marketed under the trade name TOVIAZ® by Pfizer.

Salts of fesoterodine and their preparation are reported in U.S. Pat. No. 6,858,650 and in patent application WO 2007/140986. Other forms of fesoterodine previously described include fesoterodine base, disclosed in US patent application 2006/0014832, US patent application 2010/0152483, EP 2196452, and amorphous fesoterodine fumarate, disclosed in patent application WO 2009/044278.

Polymorphism, the occurrence of different crystal forms, is a property of some molecules and molecular complexes. A single molecule may give rise to a variety of polymorphs having distinct crystal structures and physical properties like melting point, thermal behaviors (e.g. measured by thermogravimetric analysis—“TGA”, or differential scanning calorimetry—“DSC”), x-ray diffraction pattern, infrared absorption spectrum, and solid state NMR spectrum. One or more of these techniques may be used to distinguish different polymorphic forms of a compound.

Discovering new polymorphic forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need in the art for additional solid state forms of fesoterodine fumarate and of fesoterodine base and for additional methods for preparing fesoterodine fumarate and fesoterodine base crystal forms that provide fesoterodine efficiently and can be applied on an industrial scale.

### SUMMARY OF THE INVENTION

According to one embodiment the invention comprises polymorphically pure crystalline fesoterodine fumarate denoted Form A characterized by data selected from: an X-ray powder diffraction having peaks at 11.5 and 11.8 (doublet), 14.9 and 15.1(doublet), 24.1, 27.0 and 27.7 ±0.2 degrees two-theta; an X-ray powder diffraction pattern substantially as depicted in FIG. 1; a solid-state ¹³C NMR spectrum having characteristic peaks at 18.0, 127.1 and 140.5, ±0.2 ppm; a solid state ¹³C NMR spectrum having chemical shift differences between said characteristic peaks and a peak at 122.3±0.2 ppm of −104.3, 4.8 and 18.2±0.1 ppm, respectively; a solid-state ¹³C NMR spectrum substantially as depicted in FIGS. 8 and 9 and combinations thereof; wherein said polymorphically pure form contains up to 10% by weight of crystalline fesoterodine fumarate Form B as determined by using one or more XRD peaks selected from 9.7, 12.2, 14.5, 18.6 and 19.5±0.2 degrees two-theta, and contains up to 10% by weight of crystalline fesoterodine fumarate Form I as determined by using one or more solid-state ¹³C NMR peaks selected from 19.2, 58.0, 135.2, 168.3 and 170.9±0.2 ppm.

In another embodiment, the present invention comprises crystalline fesoterodine fumarate denoted Form B, characterized by data selected from: an X-ray powder diffraction pattern having peaks at 9.7, 12.2, 14.5, 18.6 and 19.5±0.2 degrees two-theta; an X-ray powder diffraction pattern substantially as depicted in FIG. 3; a solid-state ¹³C NMR spectrum having characteristic peaks at 18.9, 34.9, 48.0, 53.6, 122.5, 127.4, 135.4, and 148.8 ppm±0.2 ppm; a solid state ¹³C NMR spectrum having chemical shifts differences between said characteristic peaks and a peak at 122.5±0.2 ppm of −103.6, −87.6, −74.5, −68.9, 4.9, 12.9, and 26.3±0.1 ppm, respectively; a solid-state ¹³C NMR spectrum substantially as depicted in FIGS. 12 and 13 and combinations thereof; wherein said polymorphically pure form contains up to 10% by weight of crystalline fesoterodine fumarate Form A up to 10% by weight of crystalline fesoterodine fumarate Form I as determined by using one or more XRD peaks selected from 12.8, 18.0 and 20.8±0.2 degrees two-theta.

In another embodiment, the present invention comprises polymorphically pure crystalline fesoterodine fumarate denoted form I characterized by data selected from: an X-ray powder diffraction (XRPD) pattern having peaks at 11.8, 14.9, 16.2 and 27.4±0.2 degrees two-theta; an XRPD pattern substantially as depicted in FIG. 17; a solid-state 13C NMR spectrum having characteristic peaks at 19.2, 58.0, 122.4, 135.2, 168.3 and 170.9 ppm ±0.2 ppm; a solid state 13C NMR spectrum having chemical shifts differences between said characteristic peaks and a peak at 122.4±0.2 ppm of −103.2, −64.4, 12.8, 45.9 and 48.5±0.1 ppm, respectively; a solid-state 13C NMR spectrum substantially as depicted in FIGS. 18 and 19, and combinations thereof; wherein said polymorphically pure form contains up to 10% by weight of crystalline fesoterodine fumarate Form A as determined by using one or more XRD peaks selected from 11.5, 15.1, 24.1 and 27.7±0.2 degrees two-theta or one or more solid state 13C NMR peaks selected from 18.0, 127.1 and 140.5±0.2 ppm, and up to 10% by weight of crystalline fesoterodine fumarate Form B as determined by using one or more XRD peaks selected from 9.7, 12.2, 14.5, 18.6 and 19.5±0.2 degrees two-theta.

A crystalline form of fesoterodine fumarate, denoted Form C, is characterized by data selected from: a single crystal XRD with the following data: monoclinic crystal system; space group P2₁; unit cell parameters: a, b, c: 22.332(2), 9.3990(6), 23.0310(16) [Å], respectively, and alpha, beta, gamma: 90°, 116.493(10)°, 90°, [deg], respectively, and volume of: 4326.5(7) [Å³], Z of 6; a theoretical XRPD pattern calculated from single crystal data as depicted in FIG. 16.

In one embodiment, the present invention comprises the use of any of the above described crystalline forms of fesoterodine fumarate of the present invention for the preparation of a formulation.

In another embodiment, the present invention comprises a pharmaceutical composition comprising any one, or a combination, of the above described crystalline forms of fesoterodine fumarate of the present invention and at least one pharmaceutically acceptable excipient.

In another embodiment, the present invention comprises the use of any of the above crystalline forms as a medicament and the use of any of these crystalline forms in the manufacture of a medicament to treat overactive bladder syndrome with symptoms of urgency, urinary incontinence and increased urinary frequency.

In another embodiment, the present invention comprises a crystalline form of fesoterodine base, characterized by data selected from: an X-ray powder diffraction pattern having peaks at 8.8, 10.8, 19.0, 22.5 and 25.0±0.2 degrees two-theta; an XRPD pattern substantially as depicted in FIG. 5; an FTIR spectrum substantially as depicted in FIG. 6, and combinations thereof

In one embodiment, the present invention comprises the use of the above described polymorph of fesoterodine base to prepare fesoterodine fumarate.

In another embodiment, the present invention comprises a process for preparing fesoterodine fumarate comprising preparing the above described polymorph of fesoterodine base by the process of the present invention and converting it to fesoterodine fumarate.

### BRIEF DESCRIPTION OF THE FIGURES

FIG. 1 depicts a characteristic X-ray powder diffraction (XRPD) pattern of crystalline Fesoterodine fumarate polymorphically pure Form A according to ex.2.

FIG. 2 depicts a characteristic X-ray powder diffraction (XRPD) pattern of crystalline Fesoterodine fumarate Form B according to ex.5.

FIG. 3 depicts a characteristic X-ray powder diffraction (XRPD) pattern of crystalline Fesoterodine fumarate polymorphically pure Form B according to ex.13.

FIG. 4 depicts a characteristic X-ray powder diffraction (XRPD) pattern of crystalline Fesoterodine fumarate polymorphically pure Form I (polymorphically pure from Form B) according to ex.12.

FIG. 5 depicts a characteristic X-ray powder diffraction (XRPD) pattern of fesoterodine base according to ex.1.

FIG. 6 depicts a FT-IR spectrum of crystalline fesoterodine base according to ex.1.

FIG. 7 depicts a powder XRD pattern of Form A calculated from single crystal XRD data.

FIG. 8 depicts a solid-state ¹³C NMR spectrum of crystalline Fesoterodine fumarate polymorphically pure Form A according to ex.2.

FIG. 9 depicts a solid-state ¹³C NMR spectrum of crystalline Fesoterodine fumarate polymorphically pure Form A according to ex.2 in the range between 100-180 ppm.

FIG. 10 depicts a solid-state 13C NMR spectrum of crystalline Fesoterodine fumarate Form B according to ex.5.

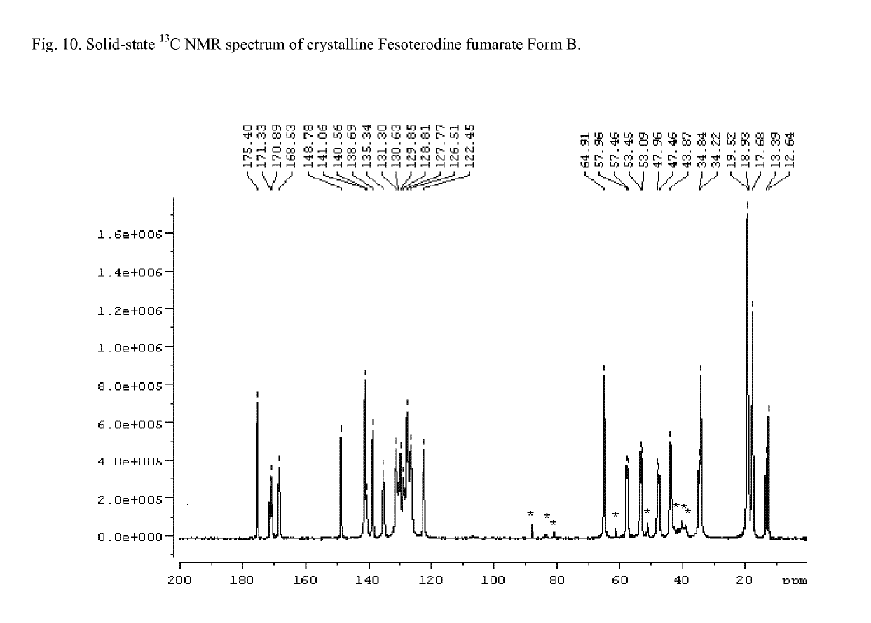

FIG. 11 depicts a solid-state ¹³C NMR spectrum of crystalline Fesoterodine fumarate Form B according to ex.5, in the range between 100-180 ppm.

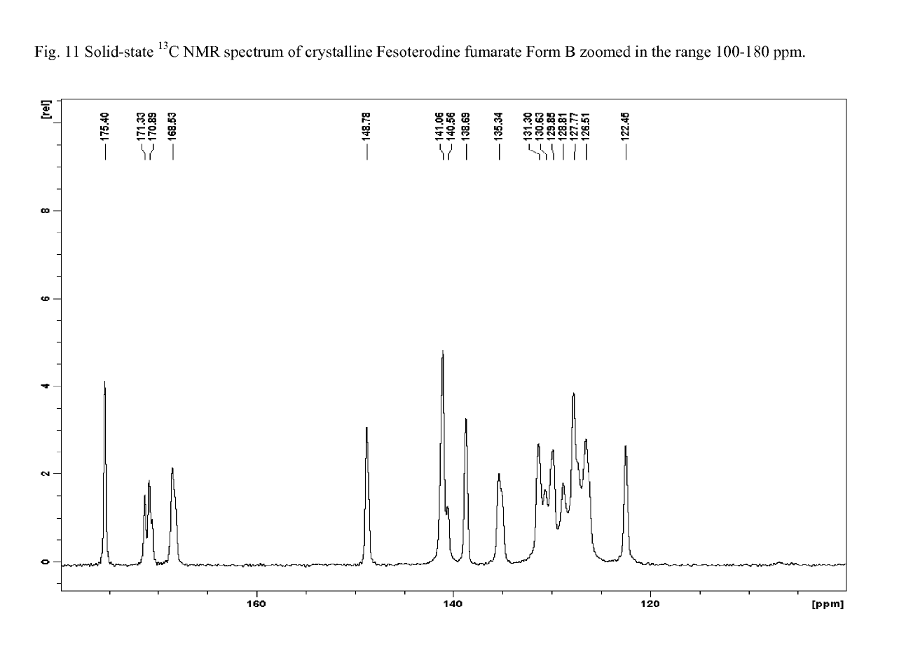

FIG. 12 depicts a solid-state ¹³C NMR spectrum of crystalline Fesoterodine fumarate polymorphically pure Form B according to ex.13.

FIG. 13 depicts a solid-state 13C NMR spectrum of crystalline Fesoterodine fumarate polymorphically pure Form B according to ex. 13 in the range between 100-180 ppm.

FIG. 14 depicts a solid-state ¹³C NMR spectrum of crystalline Fesoterodine fumarate pure Form I (polymorphically pure from Form B) according to ex.12.

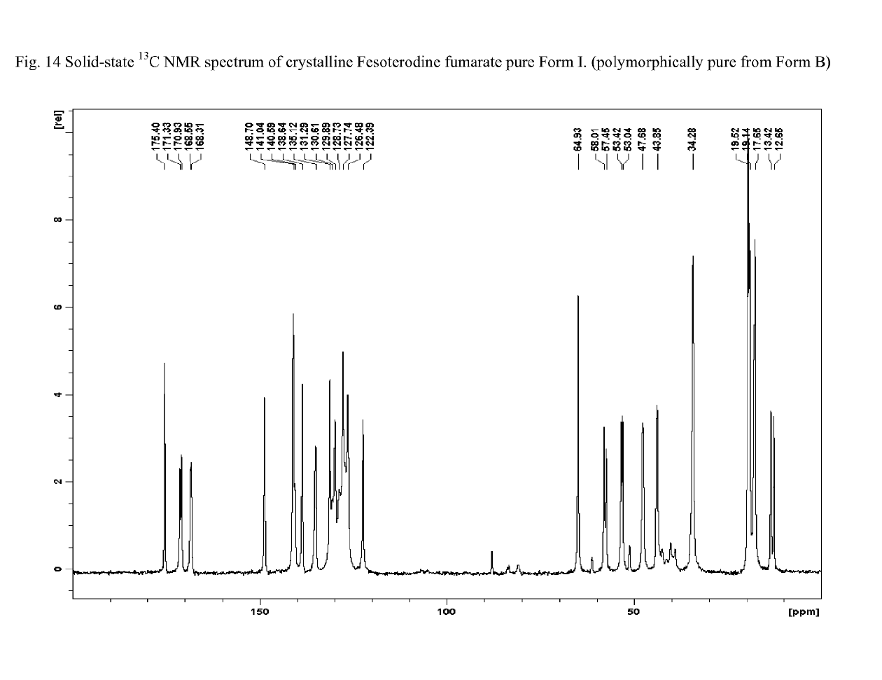

FIG. 15 depicts a solid-state ¹³C NMR spectrum of crystalline Fesoterodine fumarate pure Form I (polymorphically pure from Form B) according to ex.12 in the range between 100-180 ppm.

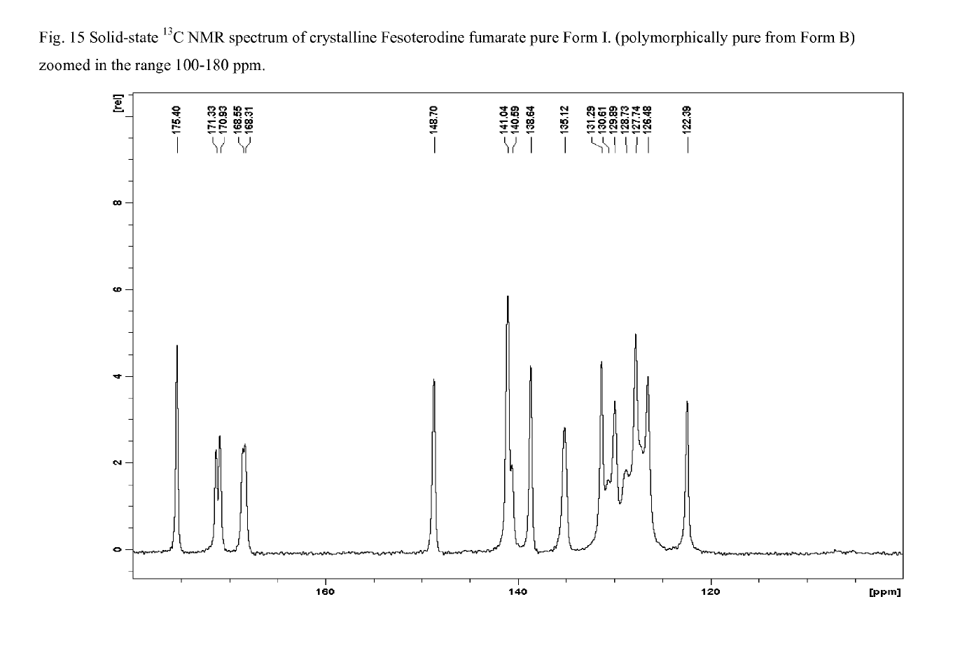

FIG. 16 depicts a powder XRD pattern of Form C according to ex.9 calculated from single crystal XRD data.

FIG. 17 depicts a characteristic X-ray powder diffraction (XRPD) pattern of polymorphically pure crystalline Fesoterodine fumarate Form I (polymorphically pure from Form A and B) according to ex.14.

FIG. 18 depicts a solid-state ¹³C NMR spectrum of polymorphically pure crystalline Fesoterodine fumarate Form I (polymorphically pure from Form A and B) according to ex.14.

FIG. 19 depicts a solid-state ¹³C NMR spectrum of polymorphically pure crystalline Fesoterodine fumarate Form I (polymorphically pure from Form A and B) according to ex.14 in the range between 100-180 ppm.

### DETAILED DESCRIPTION OF THE INVENTION

The present invention encompasses crystalline forms of fesoterodine fumarate and fesoterodine base.

A crystal form may be referred to herein as being characterized by graphical data “as depicted in” a Figure. Such data include, for example, powder X-ray diffractograms and solid state NMR spectra. The skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

The present invention relates to polymorphically pure forms of fesoterodine fumarate, preferably wherein the forms are substantially free of any other polymorph forms. Polymorphically pure therefore means 10% or less, more preferably 5% or less, most preferably 2% or less, particularly 0.2% or less. For example, between 0.2% and 10%, between 0.2% and 5% or between 0.2% and 2%.

In one embodiment the present invention encompasses a polymorphically pure crystalline form of fesoterodine fumarate, denoted herein as pure Form A.

According to one embodiment, the polymorphically pure Form A can contain up to 10%, preferably up to 5%, and more preferably up to 2% by weight of crystalline fesoterodine fumarate Form B as determined by powder X-ray diffraction, and can contain up to 10%, preferably up to 5%, and more preferably up to 2% by weight by weight of crystalline fesoterodine fumarate Form I as determined by solid state ¹³C NMR.

For example, polymorphically pure crystalline form A of fesoterodine fumarate, can contain between 0.2% and 10%, between 0.2% and 5% or between 0.2% and 2% by weight of crystalline fesoterodine fumarate denoted Form B and/or of crystalline fesoterodine fumarate denoted Form I.

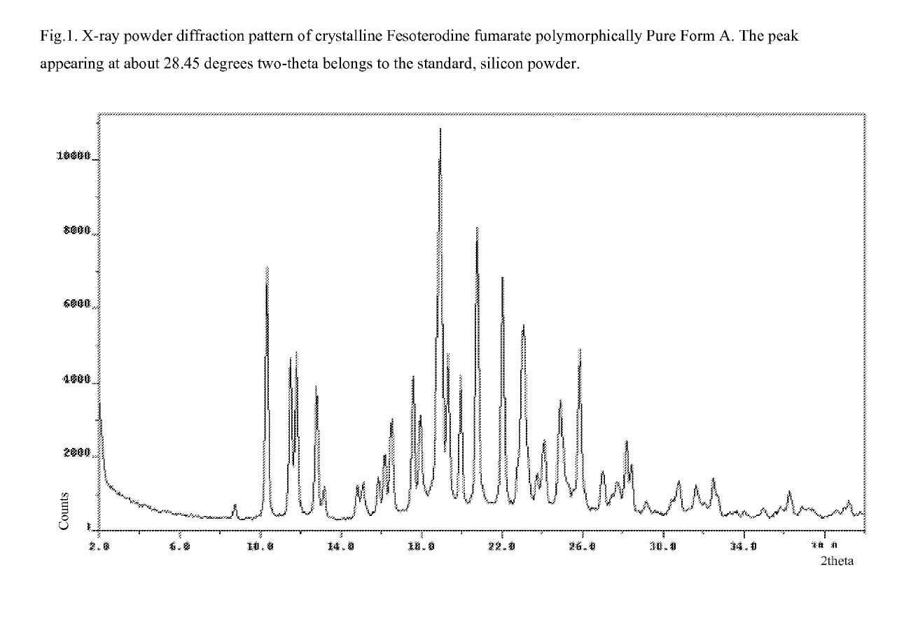

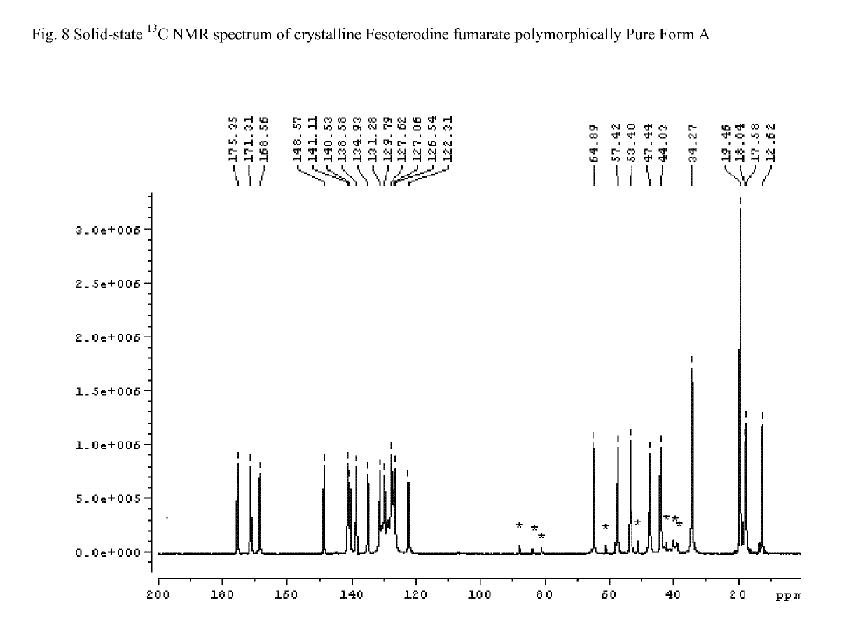

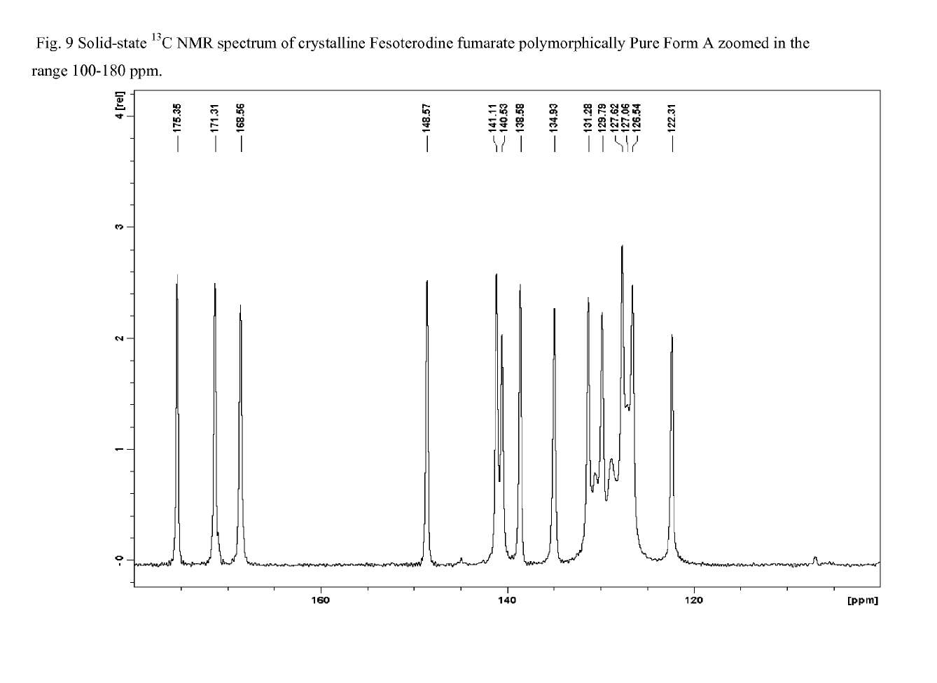

According to one embodiment, the polymorphically pure crystalline fesoterodine fumarate Form A can be characterized by data selected from: an X-ray powder diffraction having peaks at 11.5 and 11.8 (doublet), 14.9 and 15.1(doublet), 24.1, 27.0 and 27.7±0.2 degrees two-theta; an X-ray powder diffraction pattern substantially as depicted in FIG. 1; a solid-state ¹³C NMR spectrum having characteristic peaks at 18.0, 127.1 and 140.5, ±0.2 ppm; a solid state ¹³C NMR spectrum having chemical shift differences between said characteristic peaks and a peak at 122.3±0.2 ppm of −104.3, 4.8 and 18.2±0.1 ppm, respectively; a solid-state ¹³C NMR spectrum substantially as depicted in FIGS. 8 and 9 and combinations thereof. The presence and amount of crystalline fesoterodine fumarate Form B present as a polymorphic impurity can be determined using one or more XRD peaks selected from 9.7, 12.2, 14.5, 18.6 and 19.5 ±0.2 degrees two-theta. The presence and amount of crystalline fesoterodine fumarate Form I present as a polymorphic impurity can be determined using one or more solid-state ¹³C NMR peaks selected from 19.2, 58.0, 135.2, 168.3 and 170.9±0.2 ppm.

The polymorphically pure crystalline fesoterodine fumarate Form A, characterized as described above, may further characterized by additional X-ray diffraction peaks at 10.4, 12.8, 18.0, 18.9 and 20.8 ±0.2 degrees two-theta and by additional solid state ¹³C NMR peaks at 122.3, 134.9, 138.6, 148.6 and 168.6±0.2 ppm; and by the solid state ¹³C NMR chemical shifts differences between said additional ¹³C NMR peaks and the ¹³C NMR peak at 122.3±0.2 ppm of 12.6, 16.3, 26.3 and 46.3±0.1 ppm, respectively.

The data above shows that crystalline fesoterodine fumarate Form A can be characterized by a crystal structure corresponding to fesoterodine fumarate salt having a 1:1 ratio of fesoterodine to fumaric acid, i.e., one molecule of fesoterodine cation and one fumarate anion in the independent part of the unit cell.

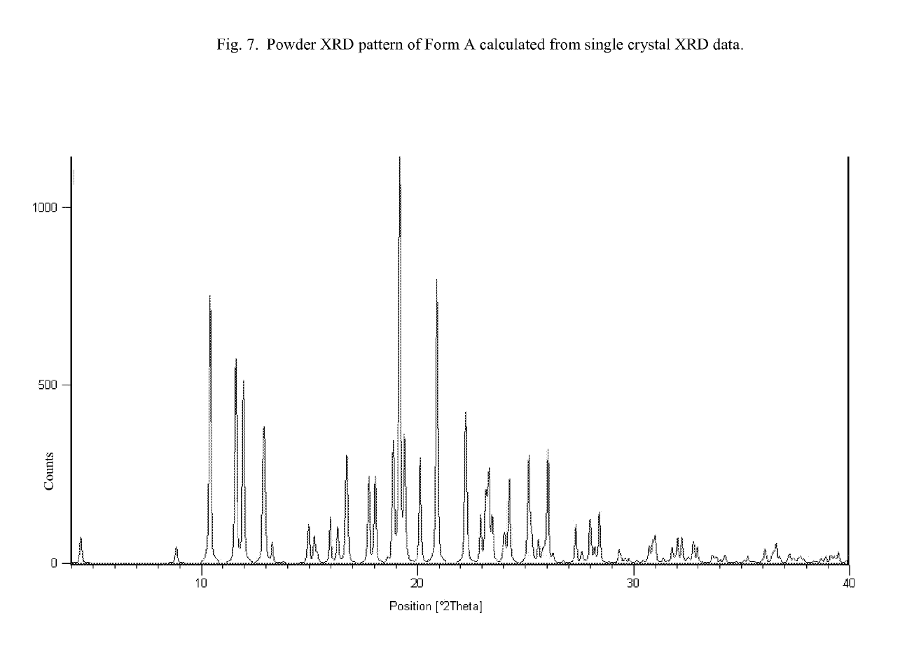

According to another embodiment, the polymorphically pure crystalline fesoterodine fumarate Form A may be characterized by data selected from: a single crystal XRD with the following data: monoclinic crystal system; space group P2₁; unit cell parameters: a, b, c: 7.6709(9), 9.3896(14), 20.099(4) [Å], respectively, and alpha, beta, gamma: 90°, 96.321(15)°, 90° [deg], respectively, and volume of: 1438.9(4) [\acute{Å}³], Z of 2 and a theoretical XRPD pattern calculated from single crystal data as depicted in FIG. 7.

According to another embodiment, the polymorphically pure crystalline fesoterodine fumarate Form A can be characterized by its melting point of 109° C. (DSC onset measured at 105.6° C., and DSC peak measured at 109.6° C.)

In another embodiment, the present invention encompasses a polymorphically pure crystalline fesoterodine fumarate, denoted herein as pure Form B.

According to one embodiment, the polymorphically pure Form B can contain up to 10%, preferably up to 5%, and more preferably up to 2% by weight of crystalline fesoterodine fumarate Form A, and can contain up to 10%, preferably up to 5%, and more preferably up to 2% by weight by weight of crystalline fesoterodine fumarate Form I as determined by powder X-ray diffraction. For example, polymorphically pure crystalline form B of fesoterodine fumarate, can contain between 0.2% and 10%, between 0.2% and 5% or between 0.2% and 2% by weight of crystalline fesoterodine fumarate denoted Form A and/or of crystalline fesoterodine fumarate denoted Form I.

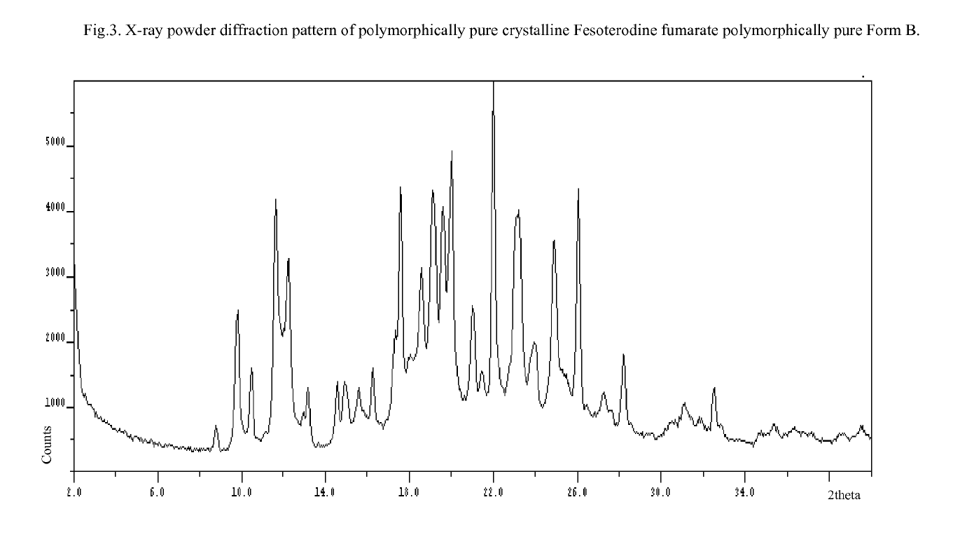

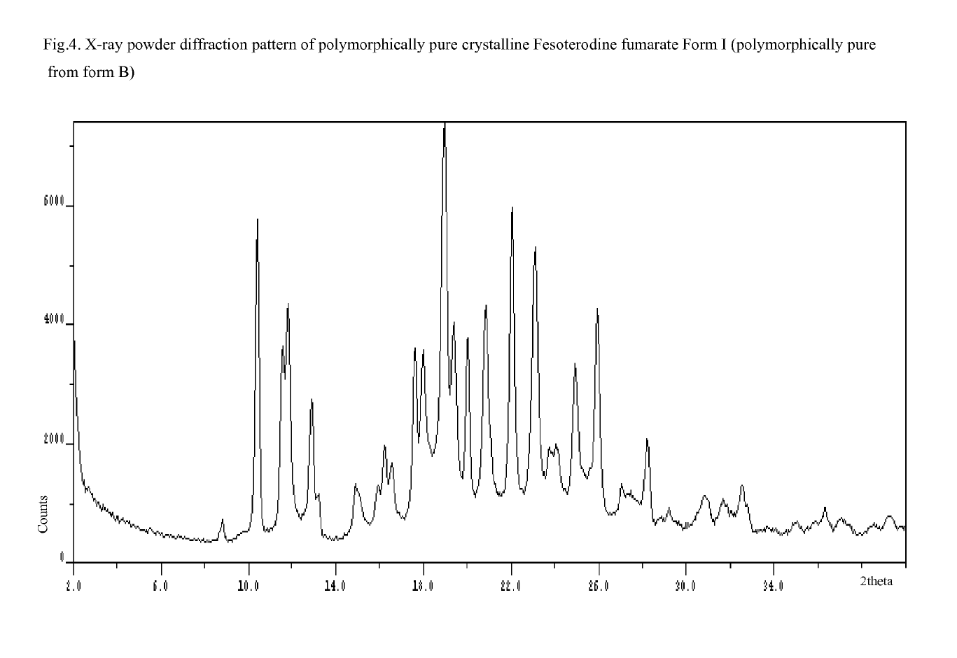

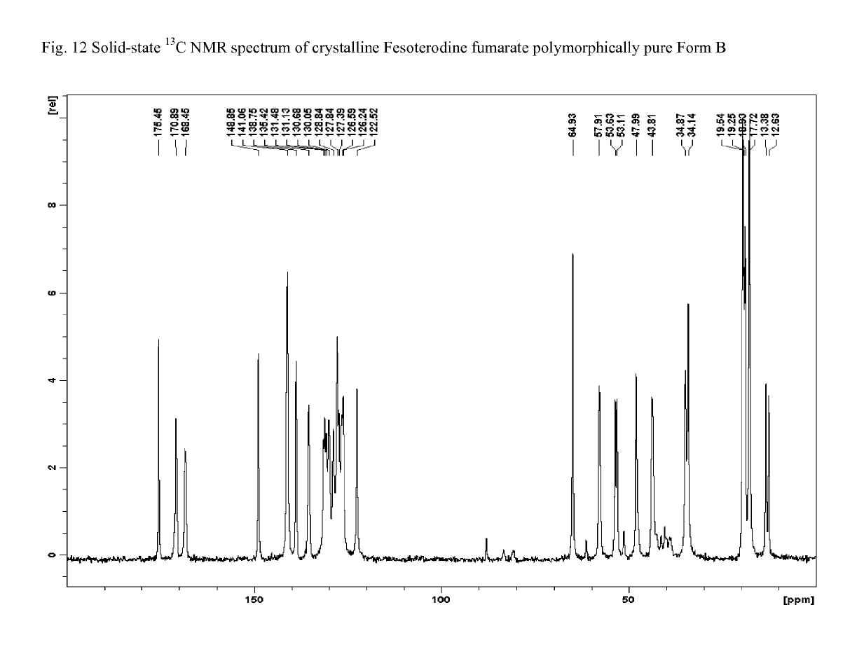

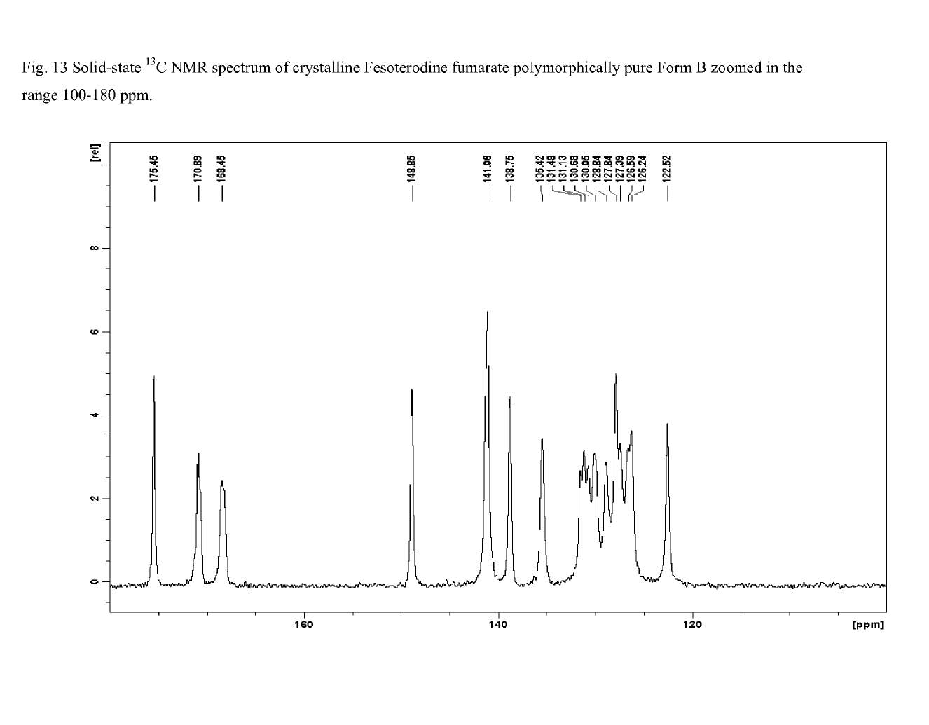

The polymorphically pure crystalline fesoterodine fumarate Form B can be characterized by data selected from: an X-ray powder diffraction pattern having peaks at 9.7, 12.2, 14.5, 18.6 and 19.5±0.2 degrees two-theta; an X-ray powder diffraction pattern substantially as depicted in FIG. 3; a solid-state ¹³C NMR spectrum having characteristic peaks at 18.9, 34.9, 48.0, 53.6, 122.5, 127.4, 135.4, and 148.8 ppm ±0.2 ppm; a solid state ¹³C NMR spectrum having chemical shifts differences between said characteristic peaks and a peak at 122.5±0.2 ppm of −103.6, −87.6, −74.5, −68.9, 4.9, 12.9, and 26.3±0.1 ppm, respectively; a solid-state ¹³C NMR spectrum substantially as depicted in FIGS. 12 and 13 and combinations thereof. The presence and amount of crystalline fesoterodine fumarate Forms A and I, present as polymorphic impurities, can be determined using one or more XRD peaks selected from 12.8, 18.0 and 20.8±0.2 degrees two-theta, an XRPD diffraction pattern substantially as depicted in FIGS. 1, 4 and 17, and combinations thereof.

The polymorphically pure crystalline fesoterodine fumarate Form B, characterized as described above, may further characterized by additional X-ray peaks at 10.4, 17.6, 20.0, 21.4 and 26.0±0.2 degrees two-theta.

In another embodiment, the present invention encompasses polymorphically pure crystalline fesoterodine fumarate denoted herein as pure Form I.

According to one embodiment, the polymorphically pure Form I can contain up to 10%, preferably up to 5%, and more preferably up to 2% by weight of crystalline fesoterodine fumarate Form A as determined by powder X-ray diffraction or solid state ¹³C NMR, and can contain up to 10%, preferably up to 5%, and more preferably up to 2% by weight by weight of crystalline fesoterodine fumarate Form B as determined by solid state ¹³C NMR. For example, polymorphically pure crystalline form I of fesoterodine fumarate, can contain between 0.2% and 10%, between 0.2% and 5% or between 0.2% and 2% by weight of crystalline fesoterodine fumarate denoted Form A and/or of crystalline fesoterodine fumarate denoted Form B. According to another embodiment, there is provided polymorphically pure crystalline fesoterodine fumarate Form I that is pure from Form B. According to yet another embodiment, there is provided polymorphically pure crystalline fesoterodine fumarate Form I that is pure from Form A and Form B.

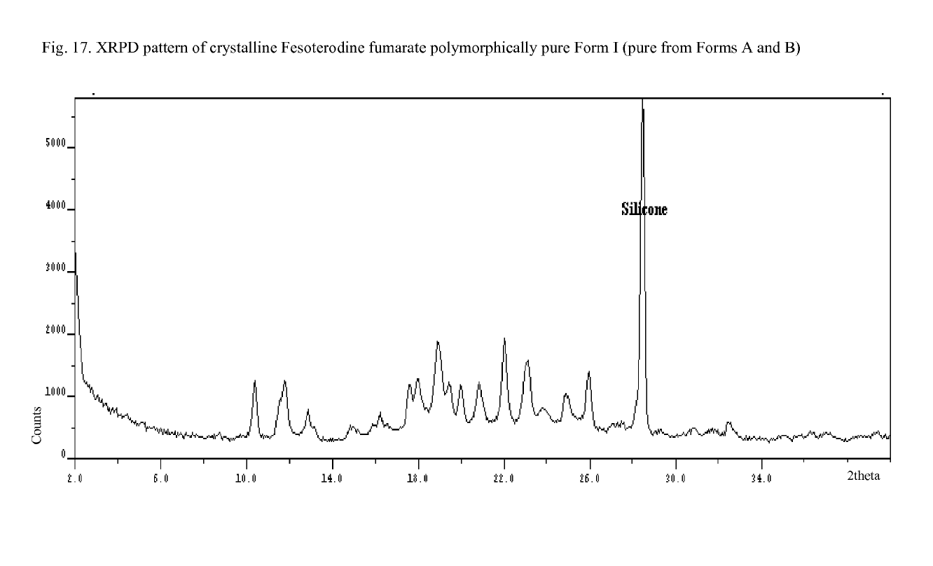

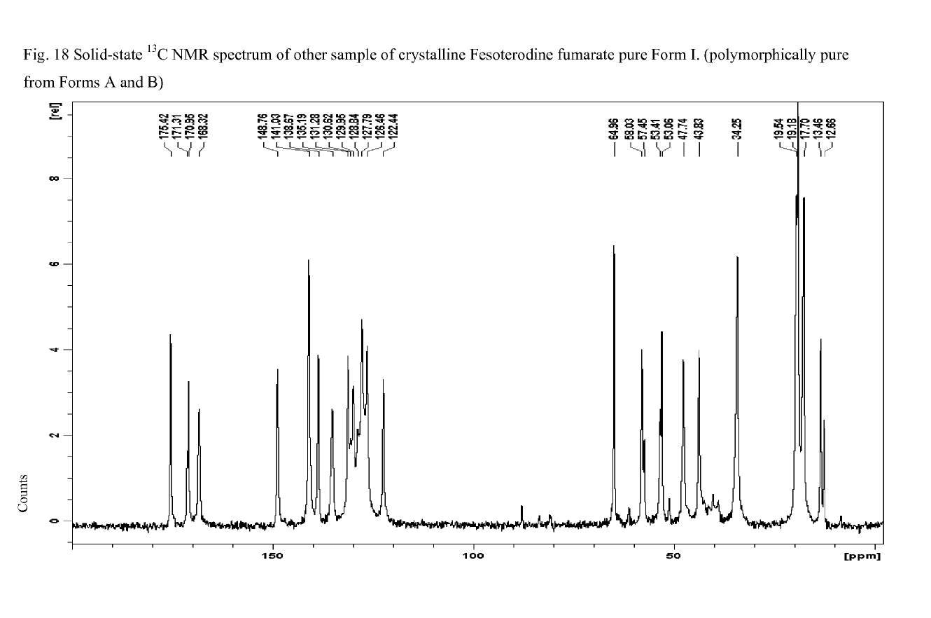

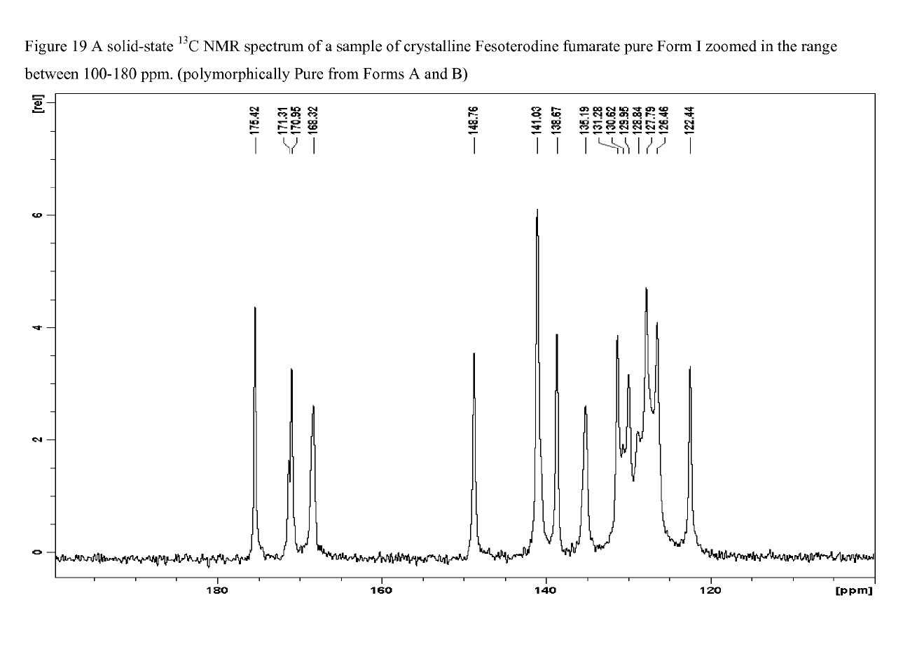

The polymorphically pure crystalline fesoterodine fumarate Form I can be characterized by data selected from: an X-ray powder diffraction (XRPD) pattern having peaks at 11.8, 14.9, 16.2 and 27.4±0.2 degrees two-theta; an XRPD pattern substantially as depicted in FIG. 17; a solid-state ¹³C NMR spectrum having characteristic peaks at 19.2, 58.0, 122.4, 135.2, 168.3 and 170.9 ppm ±0.2 ppm; a solid state ¹³C NMR spectrum having chemical shifts differences between said characteristic peaks and a peak at 122.4±0.2 ppm of −103.2, −64.4, 12.8, 45.9 and 48.5 ±0.1 ppm, respectively; a solid-state ¹³C NMR spectrum substantially as depicted in FIGS. 18 and 19, and combinations thereof. The presence and amount of Form A that may be present as a polymorphic impurity may be detected by one or more XRD peaks at 11.5, 15.1, 24.1, 27.0 27.7±0.2 degrees two-theta or by one or more solid state ¹³C NMR peaks at 18.0, 127.1 and 140.5±0.2 ppm. The presence and amount of Form B may be detected by one or more XRD peaks at 9.7, 12.2, 14.5, 18.6 and 19.5±0.2 degrees two-theta; an XRPD pattern substantially as depicted in FIGS. 1 and 2, and 3 and combinations thereof.

According to another embodiment, the present invention encompasses polymorphically pure crystalline fesoterodine fumarate Form I that is pure from Forms A and B. Pure Form I pure from Forms A and B can be characterized by data selected from: an X-ray powder diffraction (XRPD) pattern having typical peaks at 11.8, 14.9, 16.2 and 27.4±0.2 degrees two-theta and absence of diffraction peaks at 11.5, 15.1, 24.1, 27.0 and 27.7±0.2 degrees two-theta; an XRPD pattern substantially as depicted in FIG. 17; a solid-state 13C NMR spectrum having characteristic peaks at 19.2, 58.0, 122.4, 135.2, 168.3 and 170.9 ppm ±0.2 ppm; a solid state 13C NMR spectrum having chemical shifts differences between said characteristic peaks and a peak at 122.4±0.2 ppm of −103.2, −64.4, 12.8, 45.9 and 48.5±0.1 ppm respectively; a solid-state 13C NMR spectrum substantially as depicted in FIGS. 18 and 19, and combinations thereof.

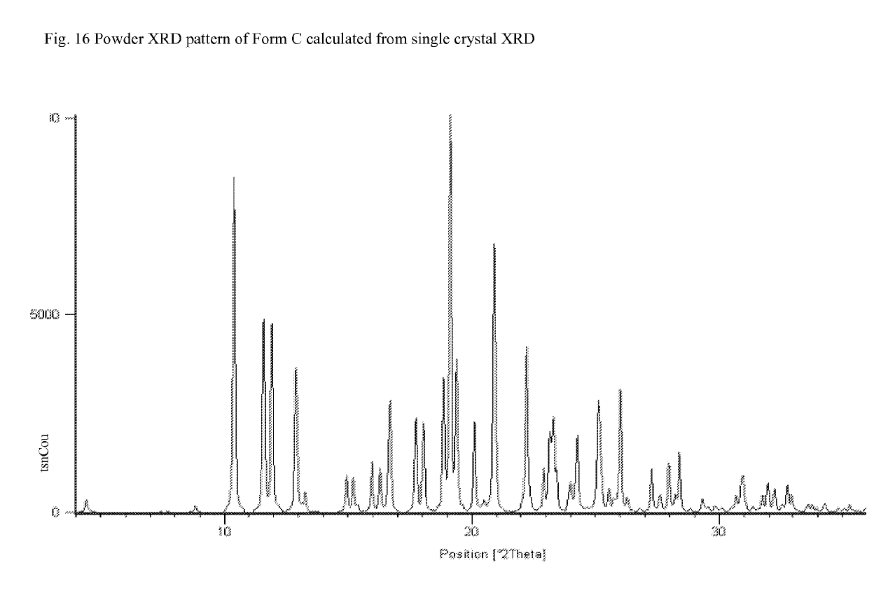

The present invention further provides a crystalline form of fesoterodine fumarate, denoted Form C. Form C is characterized by data selected from: a single crystal XRD with the following data: monoclinic crystal system; space group P2₁; unit cell parameters: a, b, c: 22.332(2), 9.3990(6), 23.0310(16) [Å], respectively, and alpha, beta, gamma: 90°, 116.493(10)°, 90°, [deg], respectively, and volume of: 4326.5(7) [\acute{Å}³], Z of 6; a theoretical XRPD pattern calculated from single crystal data as depicted in FIG. 16.

The data above shows that crystalline fesoterodine fumarate Form C can be characterized by a structure of fesoterodine fumarate salt having a ratio of 1:1 of fesoterodine to fumaric acid, i.e., three distinct symmetry-independent molecules of the fesoterodine cation and three symmetry independent molecules of the fumarate anion in the independent part of the unit cell.

The above crystalline forms of fesoterodine fumarate can be used to prepare formulations by any method known in the art. The present invention also encompasses a pharmaceutical formulation comprising any of the above crystalline forms and at least one pharmaceutically acceptable excipient.

Also encompassed by the present invention is the use of any of the above crystalline forms as a medicament and the use of any of these crystalline forms in the manufacture of a medicament to treat overactive bladder syndrome with symptoms of urgency, urinary incontinence and increased urinary frequency.

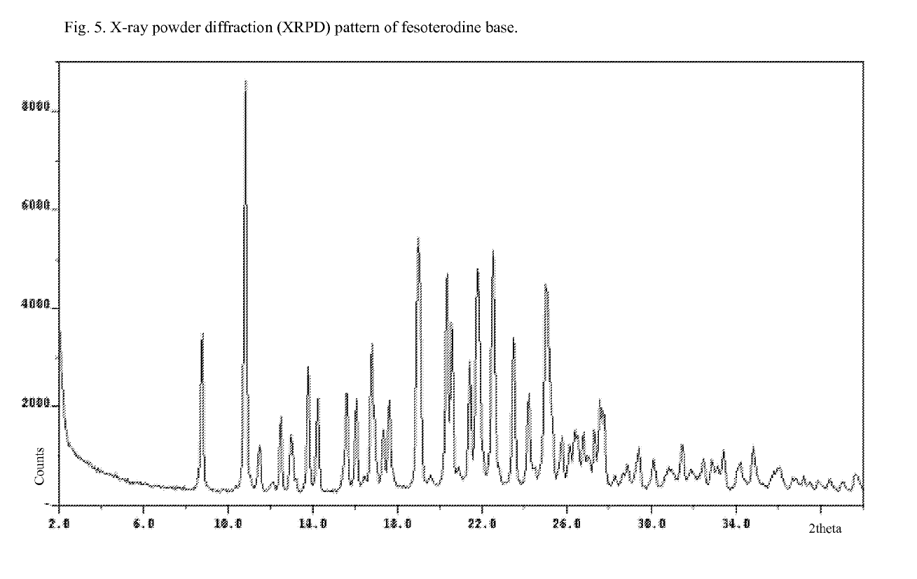

In another embodiment, the present invention comprises a crystalline form of fesoterodine base, characterized by data selected from: an X-ray powder diffraction pattern having peaks at 8.8, 10.8, 19.0, 22.5 and 25.0±0.2 degrees two-theta; an XRPD pattern substantially as depicted in FIG. 5, and combinations thereof

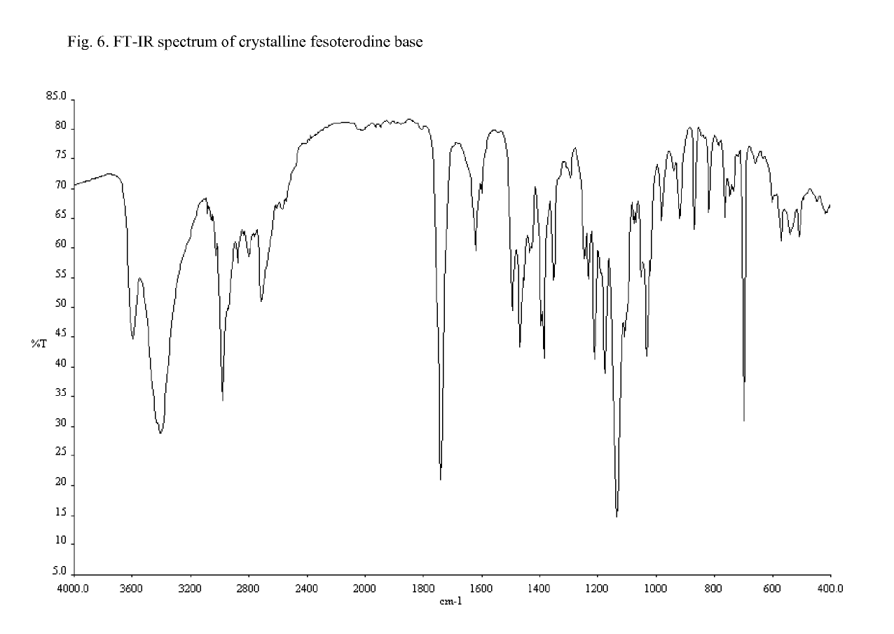

The above crystalline fesoterodine base can be further characterized by data selected from: an X-ray powder diffraction pattern having additional peaks at 13.7, 15.6, 16.8, 20.3, 21.7 and 23.5±0.2 degrees two-theta; a FT-IR spectrum having peaks at 3595, 2983, 2713, 1741, 1496, 1469, 1387, 1354, 1213, 1177, 1137, 1034, 871, 820 and 700 cm⁻¹±4 cm⁻¹; a FT-IR spectrum substantially as depicted in FIG. 6, and combinations thereof.

The above crystalline fesoterodine base can be used to prepare a fesoterodine salt, preferably, a fesoterodine fumarate salt. Such a fesoterodine salt can be prepared, for example, by a process comprising preparing the above described polymorph of fesoterodine base by the process of the present invention and converting it to a fesoterodine salt. The conversion can be done, for example, by reacting fesoterodine base polymorph with a suitable acid, such as fumaric acid.

### EXAMPLES

### PXRD

The X-ray powder diffraction patterns were measured with ARL X-ray powder diffractometer model X′TRA-030, equipped with Cu irradiation source λ=1.54056 \acute{Å}, Peltier detector, and with a round standard aluminum sample holder with round zero background quartz plate. Scanning parameters: Range: 2-40 deg. 2θ, continuous Scan, Rate: 3 deg./min. The accuracy of peak positions is defined as +/−0.2 degrees due to experimental differences like instrumentations, sample preparations.

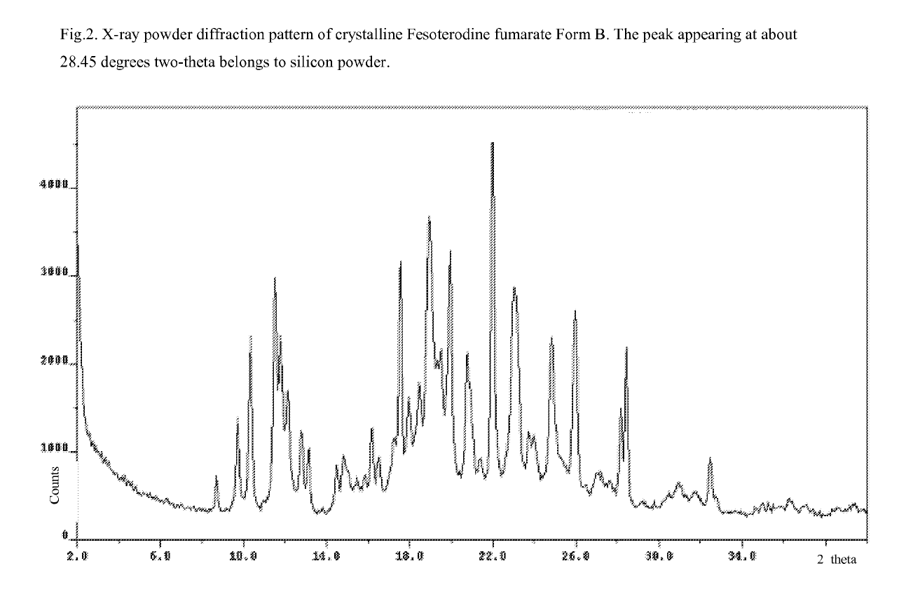

The above mentioned peak positions were determined by using silicon powder as an internal standard in an admixture with the sample measured. The position of the silicon (111) peak was corrected to be 28.45 degrees two theta. The positions of measured peaks were corrected respectively. However, no correction was performed on the diffractograms presented as FIGS. 1 and 2.

### Single Crystal X-ray Diffraction

Data was collected on Xcalibur PX Cu Kα1 (1.540598 Å) using combined φ and ω scans at 150 K. All non-hydrogen atoms were refined anisotropically. Cell parameters were determined at 150 K. References to programs used: Data collection: CrysAlisPro CCD (Oxford Diffraction, 2002); cell refinement: CrysAlisPro RED; data reduction: CrysAlisPro RED; program used to solve structure: Superflip (Palatinus & Chapuis, 2006); program used to refine structure and absolute chirality analysis: CRYSTALS (Betteridge et al., 2003); molecular graphics: Mercury, DS ViewerPro. Data export and void calculation was done by Platon (Spek, 2003).

### FT-IR Spectroscopy

Data was collected using a Perkin-Elmer Spectrum One Spectrometer, at 4 cm⁻¹ resolution with 16 scans, in the range of 4000-400 cm⁻¹. Sample was analyzed in KBr pellet. The spectrum was recorded using an empty cell as a background.

### Solid-State ¹³C NMR Spectroscopy

¹³C NMR spectra were recorded at 125 MHz using a Bruker Avance II+ 500 instrument. SB probe using 4 mm rotors. Magic angle was set using KBr. Homogeneity of magnetic field was checked using adamantine. Parameters for Cross-polarization were optimized using glycine. Spectral reference set according to glycine as external standard (176.03 ppm) for low field carboxyl signal.

Scanning parameters: Magic Angle Spinning Rate: 11 kHz

Pulse program: cp with tppm15 during decoupling

Delay time: 2 s

Number of scans: 2048

### Melting Point

USP melting point was measured by BÜCHI B-545 equipment using the following parameters: Heating rate: 1° C./min, Scan range 80-120° C.

### Example 1

### Process for Preparing Fesoterodine Base

(R)-Hydroxymethyl-tolterodine (160.0 g, 468.5 mmol) was dissolved in 6400 ml of dichloromethane (DCM) at 20-25° C. To the stirred solution, 13.0 ml of triethylamine was added. The resulting mixture was stirred for 15 min under nitrogen at 20-25° C. Isobutyryl chloride (51.0 ml dissolved in 1600 ml DCM) was added dropwise over 50 min. The resulting mixture was stirred for an additional one hour at 20-25° C. Sodium hydrogen-carbonate (79 g in 1600 ml of distilled water) was then added in one portion. This mixture was stirred vigorously for one hour. The stirring was then stopped and the organic phase was separated and concentrated at 25-30° C. under 20 mbar, resulting in isolation of 199.5 g of product as a dark yellow oil.

### Example 2

### Process for Preparing Polymorphically Pure Crystalline Fesoterodine Fumarate Form A

Fesoterodine base (199.5 g) was dissolved in 1540 ml of 3-pentanone. Fumaric acid (54.3 g) and distilled water (4.8 ml) were added. The resulting suspension was stirred for 16 hrs at 20-25° C., and then filtered. The separated solid was washed with 150 ml of 3-pentanone. The resulting white solid was dried (100 mbar, 40-45° C.) under nitrogen for 50 hrs. The obtained dry white powder (205.3 g, 83% for 2 steps) was analyzed by XRPD.

### Example 3

### Process for Preparing Polymorphically Pure Crystalline Fesoterodine Fumarate Form A

To 13.3 g of Fesoterodine base was added 30 ml of acetone and 3.4 g of fumaric acid. The resulting suspension was stirred at 40-45° C. for 0.5 hour to form a solution. To the solution was added 96 ml of 3-pentanone. This mixture was concentrated under 100 mbar of vacuum at 40-45° C. to evaporate 30 ml of the solvent. The resulting solution was cooled to 20-25° C., and seeded with crystals, which were prepared in the same way as described in Example 1. The resulting suspension was stirred for 20 hrs at 20-25° C., and then filtered. The separated solid was washed with 15 ml of 3-pentanone, and dried (100 mbar, 40-45° C.) under nitrogen for 40 hrs. The obtained dry white powder (12.7 g, 74%) was analyzed by XRPD.

### Example 4

### Process for Preparing Polymorphically Pure Crystalline Fesoterodine Fumarate Form A

Fesoterodine fumarate (1.0 g) was dissolved in 20 ml of 3-pentanone at 40-45° C. The resulting solution was cooled to 20-25° C., and stirred overnight. The suspension which formed was filtered and the separated solid was dried at 45° C. The obtained dry white powder (0.59 g, 59%) was analyzed by XRPD.

### Example 5

### Process for Preparing Crystalline Fesoterodine Fumarate Form B

Fesoterodine fumarate (3 g) was dissolved by warming in a mixture of 2-butanone (2.5 ml) and isobutyl-methyl ketone (2.5 ml). Isobutyl-methyl ketone (20 ml) was added to the mixture, and then that mixture was cooled to 20-25° C. and stirred for 16 hours. Crystals formed and were filtered off, washed with isobutyl-methyl ketone (5 ml), and air dried for 24 hours to yield 2.42 g (81% yield) of the product.

### Example 6

### Process for Preparing Crystalline Fesoterodine Fumarate Form B

Fesoterodine fumarate (0.5 g) was dissolved by warming in isobutyl-methyl ketone (20 ml). The solution was cooled to 20-25° C. and stirred for 3 hours. Crystals formed and were filtered off, washed with isobutyl-methyl ketone (5 ml), and air dried for 24 hours to yield 0.21g (42% yield) of the product.

### Example 7

### Process for Preparing Crystalline Fesoterodine Fumarate Form B

Fesoterodine fumarate (0.5 g) was dissolved in DCM (5 ml). Ethyl acetate (10 ml) was added to the solution, and then the DCM was evaporated. The thus-formed slurry was cooled to 20-25° C. and stirred for 3 hours. Crystals formed and were filtered off, washed with ethyl acetate (5 ml), and air dried for 24 hours to yield 0.35 g (70% yield) of the product.

### Example 8

### Process for Preparing Crystalline Fesoterodine Fumarate Form B

Fesoterodine fumarate (0.5 g) was dissolved in DCM (5 ml). n-Butyl acetate (5 ml) was added to the solution, and then the DCM was evaporated. The formed slurry was cooled to 20-25° C. and stirred for 3 hours. Crystals formed and were filtered off, washed with n-butyl acetate (5 ml), and air dried for 24 hours to yield 0.36 g (72% yield) of the product.

### Example 9

### Process for Preparing a Mixture of Crystalline Fesoterodine Fumarate Form A and Form C Single Crystal

Fesoterodine fumarate (150 mg) was dissolved in a mixture of isobutyl methyl ketone (7 ml) and acetone (0.9 ml) by heating to 50° C. The solution was allowed to stand in an open glass vial for 5 days. During this period the solvents were partially evaporated. Part of the fesoterodine fumarate precipitated as an oil and some crystals were formed on the interface of the precipitated oil. The crystals were isolated by filtration, and used for the crystal structure determination.

### Example 10

### Preparation of Seeding Material to Preparation of Fesoterodine Base

Fesoterodine base (appr. 0.2 g) was dissolved in dimethylcarbonate (appr. 0.1 ml). n-Heptane (appr. 2 ml) was added dropwise at room temperature while the mixture was stirred vigorously. The mixture was stirred for further 2 hrs to form a suspension.

### Example 11

### Preparation of Crystalline Fesoterodine Base

Fesoterodine base (3.7 g) was dissolved in dimethylcarbonate (15 ml), and n-heptane (15 ml) was added dropwise to the stirred solution at 20-25° C. The mixture was seeded with crystals from the suspension prepared in example 10. The mixture was stirred at 20-25° C. for 2 hrs. Additional n-heptane (10 ml) was added, and this mixture was stirred for 20 minutes. The resulting suspension was filtered, and the separated solid was washed with 5 ml of n-heptane. The prepared solid was dried under vacuum at 45° C. under nitrogen for 1 day. The obtained dry white solid (0.53 g) was analyzed by XRPD.

### Example 12

### Process for Preparing Crystalline Fesoterodine Fumarate Pure Form I

Fesoterodine base (31.4 g) was dissolved in 82 ml of 2-butanone at 20-25° C. To the stirred solution was added fumaric acid (8.86 g). The resulting suspension was stirred at 35-40 ° C., until complete dissolution. The solution was cooled to 0 ° C. To the stirred solution cyclohexane (10+10 ml) was added dropwise. After each 10 ml addition of cyclohexane, seeding crystals were added, which were prepared the same way without seeding. The vessel was allowed to warm up to 20-25 ° C., and then 20 ml cyclohexane was added dropwise. The resulting suspension was stirred for 20 minutes. An additional 42 ml of cyclohexane was added. The resulting suspension was stirred at 20-25 ° C. for 16 hrs. To the stirred suspension was added an additional 82 ml of cyclohexane, and the resulting suspension was stirred for 1 hr. Before filtration the suspension was diluted with an additional 82 ml of cyclohexane. The suspension was filtered through a G3 glass-filter, and the separated solid was washed with cyclohexane (2×60 ml). The obtained white solid was dried (40-45 ° C., 100 mbar), under nitrogen for 20 hrs. The resulting dry white solid (38.3 g, 95%) was analyzed by XRPD.

### Example 13

### Process for Preparing Crystalline Fesoterodine Fumarate Pure Form B

Fesoterodine fumarate (6 g) was dissolved by warming in a mixture of 2-butanone (5.0 ml) and isobutyl-methyl ketone (5.0 ml). Isobutyl-methyl ketone (40 ml) was added to the mixture, and the resulting mixture was cooled to 20-25° C. and stirred for 16 hours. Crystals formed and were filtered off, and air dried for 24 hours to yield 5.59 g (93% yield) of the product.

### Example 14

### Process for Preparing Crystalline Fesoterodine Fumarate Pure Form I

Fesoterodine base (8.0 g) was dissolved in 36 ml acetone. To the stirred solution 2.24 g fumaric acid was added and dissolved at 35-40° C. The clear solution was then evaporated totally. From the obtained residue, 3.2 g was dissolved in 10 ml 2-butanone. The resulting solution was divided into 4 parts. To one stirred part n-heptane (15 ml) was added dropwise while the temperature was set to 0° C. by ice/water cooling. The formed mixture was stirred vigorously at 0° C. for 4-5 hrs, settled at 2-8° C. overnight then stirred vigorously at 0° C. for an additional 2-3 hrs. The suspension was then filtered and washed with n-heptane (1-2 ml). The resulting white solid was dried at 40-45° C., to provide 430 mg of dry material.

1. Polymorphically pure crystalline fesoterodine fumarate Form A.

2. The polymorphically pure Form A according to claim 1; wherein said polymorphically pure form contains up to 10% by weight of crystalline fesoterodine fumarate Form B as determined by powder X-ray diffraction, and contains up to 10% by weight of crystalline fesoterodine fumarate Form I as determined by solid state ¹³C NMR.

3. The polymorphically pure crystalline fesoterodine fumarate according to claim 2, characterized by data selected from: an X-ray powder diffraction having peaks at 11.5 and 11.8 (doublet), 14.9 and 15.1(doublet), 24.1, 27.0 and 27.7±0.2 degrees two-theta; an X-ray powder diffraction pattern substantially as depicted in FIG. 1; a solid-state ¹³C NMR spectrum having characteristic peaks at 18.0, 127.1 and 140.5, ±0.2 ppm; a solid state ¹³C NMR spectrum having chemical shift differences between said characteristic peaks and a peak at 122.3 ±0.2 ppm of −104.3, 4.8 and 18.2±0.1 ppm, respectively; a solid-state ¹³C NMR spectrum substantially as depicted in FIGS. 8 and 9 and combinations thereof; wherein the amount of crystalline fesoterodine fumarate Form B is determined using one or more XRD peaks selected from 9.7, 12.2, 14.5, 18.6 and 19.5±0.2 degrees two-theta; and the amount of crystalline fesoterodine fumarate Form I is determined using one or more solid-state ¹³C NMR peaks selected from 19.2, 58.0, 135.2, 168.3 and 170.9±0.2 ppm.

4. The polymorphically pure crystalline fesoterodine fumarate according to claim 2, characterized by data selected from: a single crystal XRD with the following data: monoclinic crystal system; space group P2₁; unit cell parameters: a, b, c: 7.6709(9), 9.3896(14), 20.099(4) [Å], respectively, and alpha, beta, gamma: 90°, 96.321(15)°, 90° [deg], respectively, and volume of: 1438.9(4) [\acute{Å}³], Z of 2 and a theoretical XRPD pattern calculated from single crystal data as depicted in FIG. 7.

5. The polymorphically pure crystalline fesoterodine fumarate according to claim 3, further characterized by additional X-ray diffraction peaks at 10.4, 12.8, 18.0, 18.9 and 20.8±0.2 degrees two-theta and additional solid state ¹³C NMR peaks at 122.3, 134.9, 138.6, 148.6 and 168.6±0.2 ppm; and the solid state ¹³C NMR chemical shifts differences between said additional peaks and the peak at 122.3±0.2 ppm of 12.6, 16.3, 26.3 and 46.3±0.1 ppm, respectively.

6. The polymorphically pure crystalline fesoterodine fumarate according to claim 1, having up to 5% of crystalline fesoterodine fumarate denoted Form B, and up to 5% of crystalline fesoterodine fumarate denoted Form I.

7. The polymorphically pure crystalline fesoterodine fumarate according to claim 1, having up to 2% of crystalline fesoterodine fumarate denoted Form B, and up to 2% of crystalline fesoterodine fumarate denoted Form I.

8. The polymorphically pure crystalline fesoterodine fumarate according to claim 2 wherein, the crystalline is a crystalline powder having a melting point of 109° C.

9. Polymorphically pure crystalline fesoterodine fumarate Form B.

10. The polymorphically pure Form B according to claim 9; wherein said polymorphically pure form contains up to 10% by weight of crystalline fesoterodine fumarate Form A, and up to 10% by weight of crystalline fesoterodine fumarate Form I as determined by powder X-ray diffraction.

11. The polymorphically pure crystalline fesoterodine fumarate according to claim 10, characterized by data selected from: an X-ray powder diffraction pattern having peaks at 9.7, 12.2, 14.5, 18.6 and 19.5±0.2 degrees two-theta; an X-ray powder diffraction pattern substantially as depicted in FIG. 3; a solid-state ¹³C NMR spectrum having characteristic peaks at 18.9, 34.9, 48.0, 53.6, 122.5, 127.4, 135.4, and 148.8 ppm ±0.2 ppm; a solid state ¹³C NMR spectrum having chemical shifts differences between said characteristic peaks and a peak at 122.5±0.2 ppm of −103.6, −87.6, −74.5, −68.9, 4.9, 12.9, and 26.3±0.1 ppm, respectively; a solid-state ¹³C NMR spectrum substantially as depicted in FIGS. 12 and 13 and combinations thereof; wherein the amount of crystalline fesoterodine fumarate Forms A and I are determined using one or more XRD peaks selected from 12.8, 18.0 and 20.8±0.2 degrees two-theta.

12. The polymorphically pure crystalline fesoterodine fumarate according to claim 11, further characterized by additional X-ray peaks at 10.4, 17.6, 20.0, 21.4 and 26.0±0.2 degrees two-theta.

13. The polymorphically pure crystalline fesoterodine fumarate according to claim 9, having up to 5% of crystalline fesoterodine fumarate denoted Form A, and up to 5% of crystalline fesoterodine fumarate denoted Form I.

14. The polymorphically pure crystalline fesoterodine fumarate according to claim 9, having up to 2% of crystalline fesoterodine fumarate denoted Form A, and up to 2% of crystalline fesoterodine fumarate denoted Form I.

15. Polymorphically pure crystalline fesoterodine fumarate Form I.

16. The polymorphically pure Form I according to claim 15; wherein said polymorphically pure form contains up to 10% by weight of crystalline fesoterodine fumarate Form A as determined by powder X-ray diffraction or by solid state ¹³C NMR, and up to 10% by weight of crystalline fesoterodine fumarate Form B as determined by powder X-ray diffraction.

17. The polymorphically pure crystalline fesoterodine fumarate according to claim 16, characterized by data selected from: an X-ray powder diffraction (XRPD) pattern having peaks at 11.8, 14.9, 16.2 and 27.4±0.2 degrees two-theta; an XRPD pattern substantially as depicted in FIG. 17; a solid-state ¹³C NMR spectrum having characteristic peaks at 19.2, 58.0, 122.4, 135.2, 168.3 and 170.9 ppm ±0.2 ppm; a solid state ¹³C NMR spectrum having chemical shift differences between said characteristic peaks and a peak at 122.4±0.2 ppm of −103.2, −64.4, 12.8, 45.9 and 48.5±0.1 ppm, respectively; a solid-state ¹³C NMR spectrum substantially as depicted in FIGS. 18 and 19, and combinations thereof; wherein the amount of crystalline fesoterodine fumarate Form A is determined using one or more XRD peaks selected from 11.5, 15.1, 24.1 and 27.7±0.2 degrees two-theta or one or more solid state ¹³C NMR peaks selected from 18.0, 127.1 and 140.5±0.2 ppm, and the amount of crystalline fesoterodine fumarate Form B is determined using one or more XRD peaks selected from 9.7, 12.2, 14.5, 18.6 and 19.5±0.2 degrees two-theta.

18. The polymorphically pure crystalline fesoterodine fumarate according to claim 15, having up to 5% of crystalline fesoterodine fumarate denoted Form A, and up to 5% of crystalline fesoterodine fumarate denoted Form B.

19. The polymorphically pure crystalline fesoterodine fumarate according to claim 15, having up to 2% of crystalline fesoterodine fumarate denoted Form A, and up to 2% of crystalline fesoterodine fumarate denoted Form B.

20. A pharmaceutical formulation comprising any of the crystalline forms claimed in claim 1, and at least one pharmaceutically acceptable excipient.

21-23. (canceled)

24. A method of treating overactive bladder syndrome comprising administering the polymorphically pure crystalline fesoterodine fumarate of claim 1.

25. A method of treating overactive bladder syndrome comprising administering the polymorphically pure crystalline fesoterodine fumarate of claim 9.

26. A method of treating overactive bladder syndrome comprising administering the polymorphically pure crystalline fesoterodine fumarate of claim 15.
